(19) United States
(12) Patent Application Publication
(10) Pub. No.: US 2004/0219177 A1
(43) Pub. Date: Nov. 4, 2004

(54) Depleted skin barrier replenishing skin creams composition and method of application

(76) Inventor: Randy J. Jacobs, Murrieta, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/426,493

(22) Filed: Apr. 29, 2003

(51) Int. Cl. A61K 31/16; A61K 7/00

(52) U.S. Cl. 424/401; 514/625

(57) ABSTRACT

The depleted skin barrier replenishing compositions and methods of applying them involves the use of two separate skin moisturizing therapy compositions, each of which is incorporated into a pharmaceutically acceptable vehicle that may be applied in various combinations, either individually, together or sequentially with an interval of several hours between each application. The first therapeutic composition utilizes a synthetic, non-animal derived analogue of ceramide as a ceramide replacement component, and free fatty acids, in a 3:1 mole ratio. The ceramide replacement component may be bishydroxyethyl biscetyl malonamide. The second therapeutic composition utilizes a plant derived analogue of cholesterol as a cholesterol replacement component, and free fatty acids, in a 3:1 mole ratio. The cholesterol replacement component may be phytosterols.

### BACKGROUND OF THE INVENTION

The present invention relates generally to the replenishment of the human skin barrier through skin moisturization therapy, and more particularly to the use of a two-component formulation for the topical application thereof containing various combinations of lipids or their structurally similar analogues for the treatment of patients suffering from various skin diseases or disorders due to the disruption of the skin barrier function.

The human skin barrier is located in the uppermost layer of the epidermis, which is referred to as the stratum corneum. In particular, the human skin barrier has two primary physiological/physical barrier functions as an “exit” barrier and an “entrance” barrier. As an exit barrier, the skin barrier maintains skin moisturization by preventing vital water of the skin from exiting the body (i.e., evaporation of water away into the air). Conversely, as an entrance barrier, the skin barrier minimizes skin inflammation, microbial, and other foreign contaminants by preventing entrance of potential toxins, infectants, and allergens, into the body. Physiologically, in order to ensure healthy exit and entrance skin barrier performance, it is essential that the human skin barrier maintains optimal levels of lipid replenishment and molecular organization. The human skin barrier ideally retains an adequate reserve of the three naturally occurring primary skin barrier lipid components-ceramide, cholesterol and free fatty acids. These three barrier lipids are structurally organized into orderly lipid bilayers.

If the depletion of ceramide, cholesterol, and free fatty acids occur, the lipid bilayers become disorganized and the skin barrier does not properly function as it should. As a result, the skin loses water and becomes dry, cracked and fissured. Additional adverse effects of the disrupted skin barrier include the entrance of allergens and toxins that can cause the skin to become inflamed and irritated, entry of microorganisms into the body which can cause infections, increased itchiness, and development of a dry skin related rash. Skin barrier lipid depletion can lead to skin conditions such as severe dryness, itching, scratching, all of which can lead to secondary skin infections such as herpes, molluscum, warts, staphylococcus, streptococcus, pseudomonas, fungus, yeast, tuberculosis, and the like. Depleted cholesterol, ceramide, and free fatty acids are also correlated with eczema, common dry skin, excessively washed skin, and other dry and sensitive skin situations such as chapped lips, hand eczema, and leg eczema. In addition, the presence of dry ambient environmental air generally tends to aggravate such conditions. Studies have shown that it is typically younger people that suffer from ceramide depleted atopic dermatitis, while it is typically older people that suffer from cholesterol depleted dry aged skin.

Such lipid depleted skin conditions can benefit from skin moisturization therapy, in which the primary lipids, ceramide, cholesterol, and free fatty acid are restored in the skin so that the skin barrier can reorganize into its original healthy state. To accomplish this, the three lipid components are not immediately incorporated into the skin barrier lipid bilayers. Rather, the lipids first enter granular skin cells where the three lipid components are incorporated into lamellar granules, to be extruded into the intercellular skin spaces where they reorganize to form skin barrier lipid bilayers. The granular cells eventually mature into cornified cells or “corneocytes” with ceramide, cholesterol, and free fatty acid lipid bilayers interspersed between the comeocytes, to form the skin barrier structure.

With reference to the drawings, FIG. 1 depicts the progression of non-damaged, functional lipid bilayers 10 from an organized state 12, interspersed between the corneocytes 14, to a dysfunctional, damaged state 16 such that the lipid bilayers are disorganized and leaky. The depletion of ceramide, cholesterol, and free fatty acids causes the lipid bilayers to become disorganized and the skin barrier does not function as it normally should. Various environmental and physical factors such as soap, eczema, dry air, and age, play a significant role in triggering the depletion of the skin barrier. As a result, the skin loses water and becomes dry, cracked and fissured. Accordingly, there is an increased chance of allergens and toxins entering the skin barrier causing inflammation and irritation. Itchiness increases and a dry skin related rash develops. As set forth earlier, depletion of the skin barrier can lead to severe dryness, itching, scratching and secondary skin infections.

FIG. 2 depicts the beneficial effect of ceramide, cholesterol, and free fatty acid replacement moisturization therapy on the organizational structure of the replenished lipid bilayers. Through topical application of these primary lipids, adequate supplies of ceramide, cholesterol, and free fatty acids, are effectively restored, which provides impetus for the reorganization of the skin barrier into its original healthy state 12.

FIG. 3 illustrates an enlarged, schematic view of the various layers that form the epidermis 20, including the position of granular cell layers 22 which lie directly underneath the cornified cell layers 24 or corneocytes. A spinous cell layer 26 and a basal cell layer lie beneath the granular cell layers. The three primary lipid components, ceramide, cholesterol, and free fatty acids, are not immediately absorbed into the bilayers of the skin barrier upon application of topical ceramide, cholesterol, and free fatty acid containing creams to the outside skin surface. The three lipid components are first absorbed and enter granular cells of the granular cell layers where the lipids are processed, packaged, and assimilated into lamellar granules. The granular cells eventually mature into layered corneocytes with ceramide, cholesterol, and free fatty acid lipid bilayers interspersed therebetween. Thus, the corneocytes and lipid bilayers collectively form the actual skin barrier structure.

Conventional skin moisturization replacement therapy has emphasized applications of a single topical cream containing all three primary lipid components, ceramide, cholesterol, and free fatty acids. For patients in need of ceramide, a ceramide dominant cream containing all three primary lipid components, typically in a ratio of 3:1:1 of ceramide, cholesterol, and free fatty acids, is typically applied once or twice a day. Similarly, for patients in need of cholesterol, a cholesterol dominant cream containing all three primary lipid components, typically in a ratio of 1:3:1, is typically applied once or twice a day. However, such a conventional cream with all three costly components is very expensive. Most of the cost of such products may be attributed to the cost of the key lipids, ceramide and cholesterol. Therapy typically can cost $10 or more per day for a toddler with atopic dermatitis, and because such a cream is categorized as a non-drug skin moisturizer, it is currently not covered by health care insurance. Senior patients with severe skin disorders typically require two or three times as much as a toddler. Average costs of $300 or more per month for treatment of children and of $600 or more per month for adults are not uncommon.

Such conventional skin moisturizing therapy creams also typically utilize lipid components that are derived from animals. Ceramides, for example, are commonly derived from bovine brain extract, and cholesterol is commonly derived from sheep. With recent publicity of mad cow disease, anthrax, and perils of high cholesterol, many patients, if given the choice, would prefer a product not derived from animals for use on their skin.

A need therefore exists for improved methods to treat patients suffering from depleted skin barrier function through novel application of therapeutic compositions that provide significant advantages over the application of existing compositions sold on the market today. Thus, it would be desirable to provide skin moisturizing therapy compositions and methods for skin moisturizing therapy that reduce the costs of skin moisturizing therapy, avoid the use of components derived from animals, and provide for more focused, more concentrated and more effective skin moisturizing therapy. The present invention satisfies these and other needs.

### SUMMARY OF THE INVENTION

Briefly, and in general terms, in one aspect, the present invention provides for a first therapeutic composition providing skin barrier lipids for skin moisturizing therapy, utilizing a ceramide replacement component, and a second therapeutic composition utilizing a cholesterol replacement component, for replacing natural lipid bilayer components. Methods are also provided for applying a therapeutically effective amount of the two separate compositions, formulated with two lipid components of a particular molar ratio, at intervals appropriately separated in time to maximize absorption of each therapeutic composition, in varying combinations for the treatment of depleted skin barrier conditions. The invention also provides for a composition providing skin barrier lipids for skin moisturizing therapy including a ceramide replacement component and a cholesterol replacement component.

Accordingly, the invention provides for a therapeutic ceramide replacement composition and a therapeutic cholesterol replacement composition for replenishing necessary barrier lipids in a depleted human skin barrier, with each of the therapeutic compositions being incorporated in a pharmaceutically acceptable carrier or vehicle acceptable for topical application to the skin, and methods for applying the therapeutic compositions. A first therapeutic composition includes a ceramide replacement component as a major active ingredient, and one or more free fatty acids, with the molar ratio of the ceramide replacement component to the free fatty acids being approximately 3:1. A second therapeutic composition includes a cholesterol replacement component as a major active ingredient, and one or more free fatty acids, with the molar ratio of the cholesterol replacement component to the free fatty acids being 3:1. Exemplary free fatty acids that may be used include the essential free fatty acids, such as linoleic acid, linolenic acid, and arachidonic acid, and non-essential free fatty acids, such as palmitic acid, stearic acid, oleic acid, and docosanoic acid, for example. Types of pharmaceutically acceptable carriers or vehicles acceptable for topically applying each therapeutic composition to the skin include creams, gels, lotions and ointments.

In accordance with the invention, in one preferred embodiment, a synthetic, non-animal derived analogue of ceramide, such as bishydroxyethyl biscetyl malonamide, is used in the first therapeutic composition as the ceramide replacement lipid component, and a plant-derived analogue of cholesterol, such as plant sterols, commonly known as phytosterols, is used as the cholesterol replacement lipid component. Other synthetic, non-animal derived ceramide replacement compositions may also be suitable for use as the ceramide replacement composition, and other non-animal derived cholesterol replacement compositions may also be used as the cholesterol replacement component.

The invention also provides for a composition providing skin barrier lipids for skin moisturizing therapy including both a synthetic, non-animal derived analogue of ceramide, such as bishydroxyethyl biscetyl malonamide, as the ceramide replacement component, and a plant-derived analogue of cholesterol, such as plant sterols, commonly known as phytosterols, as described above, as the cholesterol replacement component, as well as one or more free fatty acids. In one such composition the molar ratio of the ceramide replacement component, the cholesterol replacement component, essential free fatty acids and non-essential free fatty acids is approximately 3:1:1:1. In another such composition the molar ratio of the ceramide replacement component, the cholesterol replacement component, essential free fatty acids and non-essential free fatty acids is approximately 1:3:1:1. In another such composition the molar ratio of the ceramide replacement component, the cholesterol replacement component, essential free fatty acids and non-essential free fatty acids is approximately 1:1:1:1.

In another aspect of the invention, a method for replenishing necessary lipids in a depleted human skin barrier through application of at least one therapeutic composition to the skin is disclosed. Specifically, a therapeutically effective amount of a therapeutic composition incorporated into a pharmaceutically acceptable carrier or vehicle is topically applied to the skin. The therapeutic composition may be either one of the two lipid component compositions having a 3:1 molar ratio as described above. For example, patients in need of ceramide may use the ceramide replacement composition described above containing two lipid components, the ceramide replacement component as the major active ingredient, and free fatty acids as the minor active ingredient, once a day or once every other day to replenish lost ceramides. Patients in need of cholesterol may use the cholesterol replacement composition described above, containing two lipid components, the cholesterol replacement component as the major active ingredient, and free fatty acids as the minor active ingredient, incorporated into a pharmaceutically acceptable carrier or vehicle, once a day or once every other day to replenish lost cholesterol.

In another aspect of the invention, a method for replenishing necessary barrier lipids in a depleted human skin barrier through use of two separate compositions being applied sequentially is disclosed. The first and second therapeutic compositions of lipid components having a 3:1 molar ratio of the major component and essential/non-essential fatty acids, respectively, are the same as set forth above. In one preferred embodiment, the two separate therapeutic compositions are applied topically to a patient's skin one after the other following a suitable interval of time. Typically a therapeutically effective amount of the first therapeutic composition incorporated into a pharmaceutically acceptable vehicle is topically applied to the skin, and after a period of several hours such as about twelve hours has elapsed, a therapeutically effective amount of the second therapeutic composition incorporated into a pharmaceutically acceptable vehicle is topically applied to the skin.

Other features and advantages of the present invention will become more apparent from the following detailed description of the invention when taken in conjunction with the accompanying exemplary drawings.

### BRIEF DESCRIPTION OF THE DRAWINGS

FIG. 1 is an illustration of the effect of certain factors on the organization of epidermal lipid bilayers and subsequent depletion of the human skin barrier or stratum corneum.

FIG. 2 is an illustration of epidermal lipid bilayers organized between comeocytes.

FIG. 3 is an enlarged illustration of the various layers of the epidermis, including the granular cell layer and the cornified cell layer.

FIG. 4 is a schematic illustration of the molecular structure of bishydroxyethyl biscetyl malonamide, a synthetic, non-animal derived analogue of ceramide.

FIG. 5 is an illustration of the molecular structure β-sitosterol.

FIG. 6 is an illustration of the molecular structure of stigmasterol.

FIG. 7 is an illustration of the molecular structure of campesterol.

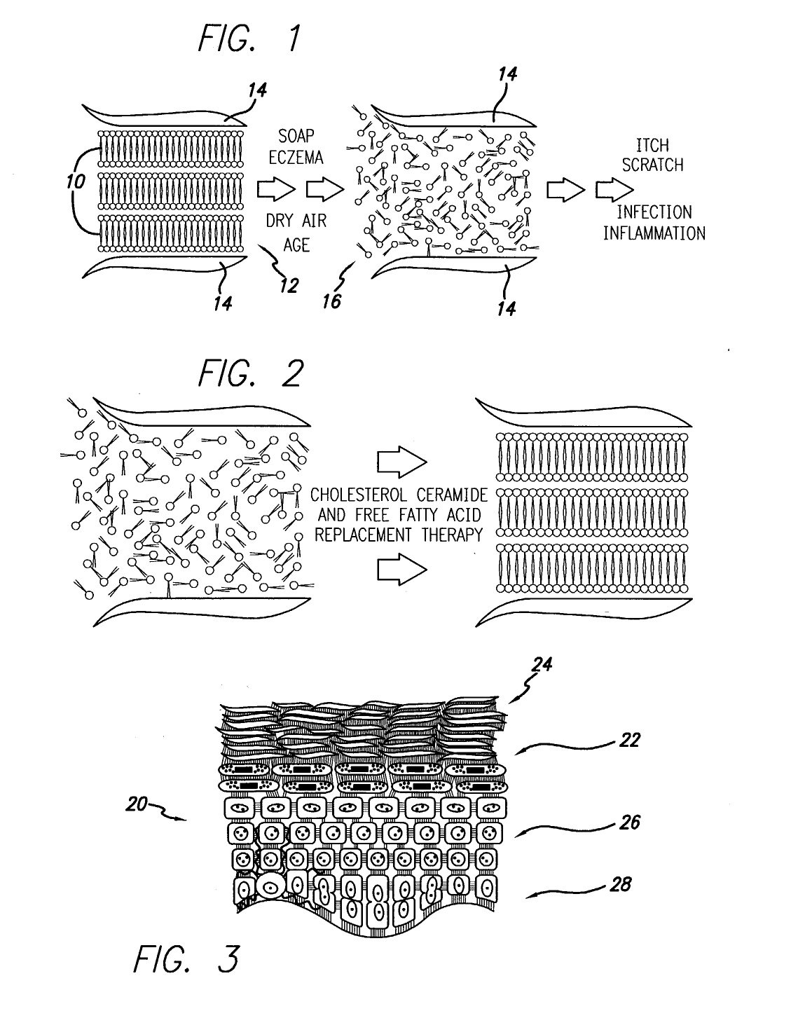

### DETAILED DESCRIPTION OF THE PREFERRED EMBODIMENTS

The present invention is directed to improved methods for replenishing necessary barrier lipids in a depleted human skin barrier. In particular, the present invention improves upon the prior art through the introduction of the use of two separate compositions (i.e., creams) for application of a ceramide replacement composition and a cholesterol replacement composition separately, rather than in one single composition, for more focused, more concentrated and more effective skin moisturizing therapy. The two new compositions are two-component formulations for replenishing depleted lipids of the skin barrier. These new compositions include a 3:1 molar ratio of a ceramide replacement component and free fatty acids, and a 3:1 molar ratio of cholesterol and free fatty acids, which can each be applied singly for treatment of particular conditions, or which can be applied alternatingly, separated by a time interval of several hours between applications, such as about twelve hours, for example.

In accordance with the invention, a first therapeutic composition is provided that includes the active ingredients of a ceramide replacement component and essential and/or nonessential free fatty acids, with the molar ratio of ceramide replacement component to the free fatty acids being about 3:1. Exemplary free fatty acids that may be used include the essential free fatty acids, such as linoleic acid, linolenic acid, and arachidonic acid, and non-essential free fatty acids, such as palmitic acid, stearic acid, oleic acid, and docosanoic acid, for example. The percent composition of the ceramide replacement component in the first therapeutic composition is about 1-2%, and the percent composition of free fatty acids in the first therapeutic composition is about 0.33-66%, with the remainder of the composition consisting essentially of a pharmaceutically acceptable carrier suitable for topical application to a patient's skin. A second therapeutic composition is also provided that includes a cholesterol replacement component and essential and/or nonessential free fatty acids, with the molar ratio of cholesterol to the free fatty acids being about 3:1. The percent composition of cholesterol in the second therapeutic composition is about 2-3%, and the percent composition of free fatty acids in the second therapeutic composition is about 0.66-1%, with the remainder of the composition consisting essentially of a pharmaceutically acceptable carrier suitable for topical application to a patient's skin. The first and second compositions can be applied singly for treatment of corresponding ceramide-depletion skin conditions or cholesterol-depletion skin conditions. Because free fatty acids are easier to replenish, less of the free fatty acids are required than the other principal lipid components. Thus, the free fatty acid molar concentration is kept constant at about one mole throughout the day.

In accordance with one embodiment of the present invention, a therapeutically effective amount of the first therapeutic composition incorporated into a pharmaceutically acceptable carrier or vehicle is topically applied to the patient's skin. After a duration of several hours, for example, such as about twelve hours, a therapeutically effective amount of the second therapeutic composition incorporated into a pharmaceutically acceptable carrier or vehicle is topically applied to the patient's skin.

It should be appreciated that it is not meant by the foregoing that the present invention should be limited to the preceding specific embodiment described herein, since the first and second therapeutic compositions can be easily interchanged for application in either the morning or evening hours of the day as desired. For example, an older dry skin patient may desire to initiate treatment by applying the cholesterol dominant composition in the morning followed by the ceramide dominant composition about twelve hours later.

As part of this novel method of replenishing the human skin barrier, the application of each cream is separated by about twelve hours with ceramide replacement therapy emphasized at one time and cholesterol replacement therapy emphasized at another time. This improved method advantageously minimizes cost to the patient, while focusing and concentrating the skin barrier therapy. The two-component cream compositions of the present invention effectively decrease the amount of expensive animal-derived ceramides and cholesterol consumed in a twenty-four hour period and increase the effectiveness of the skin barrier therapy. Depending on the quantity of ceramide and cholesterol required by individual patient needs, expenditures are also reduced by eliminating unnecessary application of any unneeded ceramide or cholesterol component.

Regarding another advantage of this novel method of replenishing the human skin barrier, it is known that some patients require both ceramide and cholesterol. For example, the average adult diabetic with dry skin will need both ceramide and cholesterol. However, other patients may not need both components-not all patients need cholesterol and not all patients need ceramide. This is particularly true for seniors who need mostly cholesterol and children who need mostly ceramide. With the two cream two-component compositions, the patient does not have to pay out-of-pocket for an unneeded component.

With the improved methods and compositions of the present invention, the human granular skin cells are given a chance to absorb and assimilate one ceramide replacement component or cholesterol replacement component at a time rather than both at the same time. The ceramide replacement component and the cholesterol replacement component do not have to compete with each other for absorption. Accordingly, the specific lipid infusion is more focused, concentrated, and effective. As a result, both ceramide replacement and cholesterol replacement components, in maximum concentrations, are applied and absorbed typically within a twenty-four hour period of time to complete the necessary replenishment, yet each lipid component is allowed its own particular dedicated span of time for granular cell absorption. The net result is more effective utilization of costly lipid ingredients, quicker symptomatic relief, and even greater cost reduction by decreasing healing time and the amount of lipid replacement cream necessary to see beneficial results.

One example of dry skin therapy in accordance with the present invention is as follows. A patient suffering from dry skin applies a ceramide replacement component-free fatty acid cream in the morning followed by a cholesterol replacement component-free fatty acid cream in the evening about twelve hours later. In the morning, the skin would be able to absorb and assimilate the ceramide replacement component, and in the evening the skin would be able to absorb and assimilate the cholesterol replacement component. This new moisturization method allows for a higher total effective concentration of the optimal 3:1 molar ratio of the ceramide replacement component to free fatty acids to absorb and assimilate in the morning and a higher total effective concentration of the optimal 3:1 molar ratio of the cholesterol replacement component to free fatty acids to absorb and assimilate in the evening. Overall skin barrier restoration is hence much improved with respect to healing time and effective lipid replenishing.

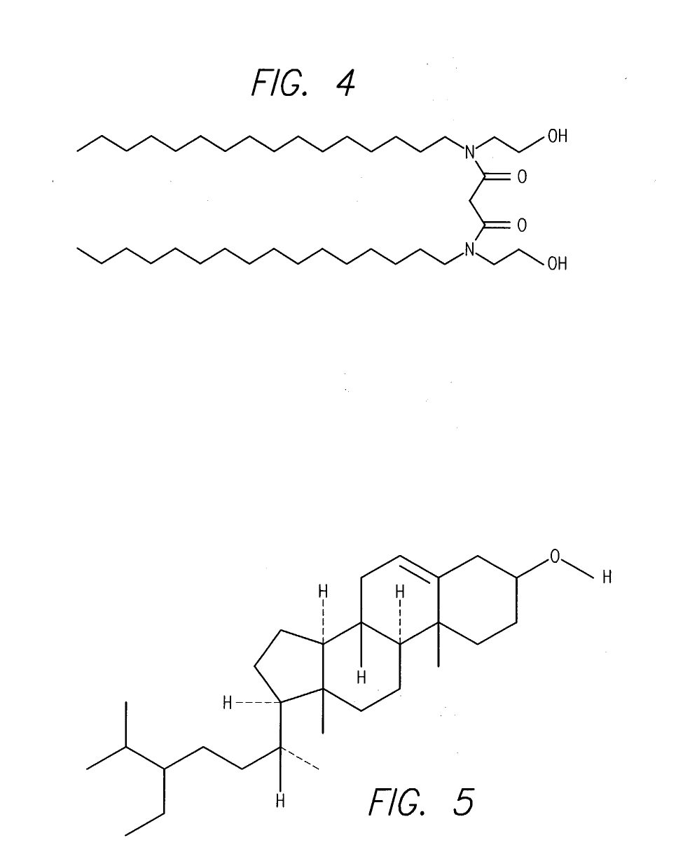

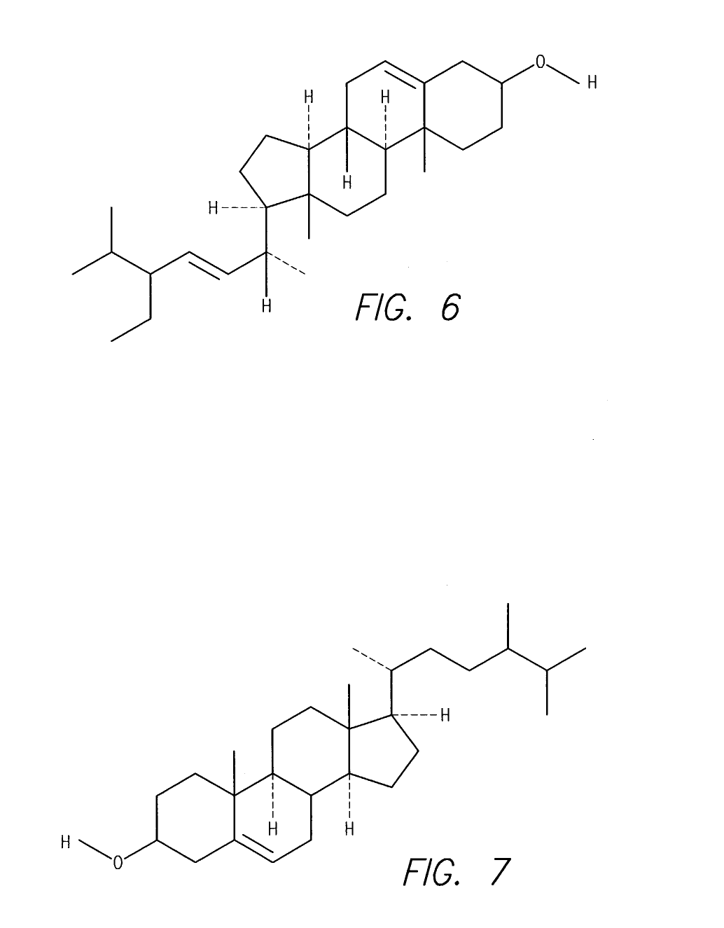

As an alternative to animal derived lipids, the novel two-component ceramide replacement composition of the present invention contains and utilizes a synthetic, non-animal derived analogue of ceramide as the ceramide replacement component, such as bishydroxyethyl biscetyl malonamide, available from Quest International, England, under the trade name Questamide H, although other synthetic, non-animal derived ceramide replacement compositions may also be suitable. FIG. 4 depicts the molecular structure of bishydroxyethyl biscetyl malonamide, the synthetic, non-animal derived analogue of ceramide. The two component cholesterol replacement composition advantageously contains a plant-derived analogue of cholesterol, such as soy plant sterols, commonly known as phytosterols, available, for example, from NutriScience Innovations, LLC, Fairfield, Conn., although other similar non-animal derived cholesterol analogues may also be suitable. FIGS. 5, 6 and 7 depict the molecular structure β-sitosterol, stigmasterol and campesterol, principal plant sterols covered by the term “phytosterols.” However, the phytosterols may also include brassicasterol and stanols, such as sitosterol, for example, which are hydrogenation products of the respective plant sterols.

Table 1 set forth below summarizes various chemical properties of these principal ceramide and cholesterol based analogues.

TABLE 1
Chemical Properties of Select Analogues of
Ceramide and Cholesterol

| INCI Name [Empirical | | |
|---|---|---|
| Formula] | Trade Name | Molecular Weight (MW) |
| Soybean (glycine soja) | Phytosterol | Refined natural oils of |
| sterol [β-Sitosterol: | | β-Sitosterol (MW = 415); |
| C₂₉H₅₀O; Stigmasterol: | | Stigmasterol (MW = 413); |
| C₂₉H₄₈O; Campesterol: | | Campesterol (MW = 401) |
| C₂₈H₄₈O] | | |
| Bishydroxyethyl biscetyl | Questamide H | MW = 638 |
| malonamide [C₃₉H₇₈N₂O₄] | | |

The incorporation of non-animal analogues of the primary lipids, ceramide and cholesterol, for use in the two-component compositions of the present invention, decreases the concern that some patients may feel regarding possible animal transmitted infections such as mad cow disease and anthrax. The plant-derived option also eases the concern of those patients with high serum cholesterol hesitant to use cholesterol on their skin. Further, the use of synthetic and plant-derived ingredients provides a price advantage. The cost of animal derived ingredients depends largely on the availability of animals needed for the ingredient and the costs of handling. For example, cholesterol depends on sheep from Australia while ceramide depends on the availability of inspected brain tissue. In order to prevent transport of infectious disease, animal products must be certified and tested before transporting into or out of countries. Synthetic and plant derived ingredients are not affected by these cost elevating factors.

The term “therapeutically effective amount” as used in the context of the present invention refers to any amount that will provide a substantial relief of symptoms as a result of being consistently applied to the affected area of the skin over time. It can be appreciated that the optimum amounts of the two-component compositions used for treating depleted human skin barrier and achieve the desired effect will be readily apparent to those skilled in the art. Exemplary of types of pharmaceutically acceptable vehicles for topically applying the therapeutic composition include water-based emulsions, creams, gels, lotions, and ointments. Currently preferred ingredients in a carrier or vehicle for topically applying the therapeutic composition of the invention include water, glycerin, sodium PCA, petrolatum, mineral oil, and combinations thereof.

In another embodiment of the present invention, one two-component composition may be applied directly after the other one to the patient's skin in varying ratios appropriate to a patent's needs. This sequential application of the first and second two-component compositions such that the compositions are used together in ratios appropriate to the patient is an alternative to treatment of depleted skin barrier without a separation of several hours between each application. The molar ratios of each two-component lipid composition are the same as in the preceding embodiment. The patient derives a benefit from the use of non-animal derived analogues of the lipids, ceramide and cholesterol, as described above.

In a further embodiment of the present invention, depending on the patient's needs in terms of moisturization therapy, one specific lipid dominant composition may be used either once a day or once every other day as needed. For example, for patients in need of ceramide, the ceramide dominant (3:1) composition containing the two components of ceramide and free fatty acid is used once a day to replenish lost ceramide. Similarly, for patients in need of cholesterol, the cholesterol dominant (3:1) composition containing the two components of cholesterol and free fatty acid is used once a day to replenish lost cholesterol. The molar ratios of the two-component lipid compositions are the same as in the preceding embodiments. Through use of non-animal derived analogues of ceramide and cholesterol, such as bishydroxyethyl biscetyl malonamide and phytosterols, the patient derives a benefit previously unavailable to them from existing products sold on the market.

In an alternate embodiment, the invention also provides for compositions providing skin barrier lipids for skin moisturizing therapy including both the ceramide replacement component and the cholesterol replacement component along with free fatty acids. The ceramide replacement component is a synthetic, non-animal derived analogue of ceramide, such as bishydroxyethyl biscetyl malonamide, and the cholesterol replacement component is a plant-derived analogue of cholesterol, such as plant sterols, commonly known as phytosterols, as described above. For example, in one such composition the molar ratio of the ceramide replacement component, the cholesterol replacement component, essential free fatty acids and non-essential free fatty acids is approximately 3:1:1:1. In another such composition the molar ratio of the ceramide replacement component, the cholesterol replacement component, essential free fatty acids and non-essential free fatty acids is approximately 1:3:1:1. In another such composition the molar ratio of the ceramide replacement component, the cholesterol replacement component, essential free fatty acids and non-essential free fatty acids is approximately 1:1:1:1.

While the invention has been described in connection with certain disclosed embodiments, it is not intended to limit the scope of the invention to the particular forms set forth herein, but, on the contrary, it is intended to cover all such alternatives, modifications, and equivalents as may be included in the spirit and scope of the invention as defined by the appended claims.

What is claimed:

1. A method for replenishing necessary barrier lipids in a patient's depleted skin barrier, comprising:
topically applying to the patient's skin a therapeutically effective amount of a first therapeutic composition incorporated into a pharmaceutically acceptable carrier, the therapeutic composition consisting essentially of a ceramide replacement component as a major active ingredient, and at least one free fatty acid selected from the group of essential free fatty acids and non-essential free fatty acids, the molar ratio of the ceramide replacement component to the at least one free fatty acid being about 3:1; and
topically applying to the patient's skin a therapeutically effective amount of a second therapeutic composition incorporated into a pharmaceutically acceptable carrier, the second therapeutic composition consisting essentially of a cholesterol replacement component, and at least one free fatty acid selected from the group consisting of essential free fatty acids and non-essential free fatty acids, the molar ratio of the cholesterol replacement component to the at least one free fatty acid being about 3:1.

2. The method of claim 1, wherein said second therapeutic composition is topically applied to the patient's skin after a duration of at least several hours from the application of the first therapeutic composition.

3. The method of claim 1, wherein the ceramide replacement component is a synthetic, non-animal derived analogue of ceramide.

4. The method of claim 3, wherein the synthetic, non-animal derived analogue of ceramide is bishydroxyethyl biscetyl malonamide.

5. The method of claim 1, wherein the cholesterol replacement component is a plant-derived analogue of cholesterol.

6. The method of claim 5, wherein the plant-derived analogue of cholesterol is at least one plant sterol.

7. The method of claim 5, wherein the plant-derived analogue of cholesterol is selected from the group consisting of phytosterols.

8. The method of claim 1, wherein the essential and non-essential free fatty acids are selected from the group consisting of arachidonic acid, linoleic acid, linolenic acid, palmitic acid, stearic acid, oleic acid, and docosanoic acid.

9. The method of claim 1, wherein the percent concentration of the ceramide replacement component of the therapeutic composition in the pharmaceutically acceptable carrier is about 1-2%.

10. The method of claim 1, wherein the percent concentration of the at least one free fatty acid of the first therapeutic composition in the pharmaceutically acceptable carrier is about 0.33-66%.

11. The method of claim 1, wherein the percent concentration of the cholesterol replacement component of the second therapeutic composition is about 2-3%.

12. The method of claim 1, wherein the percent concentration of the at lest one free fatty acid of the second therapeutic composition in the pharmaceutically acceptable carrier is about 0.66-1%.

13. The method of claim 1, wherein the pharmaceutically acceptable carrier is suitable for topically applying the therapeutic composition to the skin and is selected from the group of creams, gels, lotions and ointments.

14. A method for replenishing necessary barrier lipids in a patient's depleted skin barrier, comprising:
topically applying to the patient's skin a therapeutically effective amount of one of a first therapeutic composition and a second therapeutic composition, said first therapeutic composition being incorporated into a pharmaceutically acceptable carrier, said therapeutic composition consisting essentially of a ceramide replacement component as a major active ingredient, and at least one free fatty acid selected from the group of essential free fatty acids and non-essential free fatty acids, the molar ratio of the ceramide replacement component to the at least one free fatty acid being about 3:1, said second therapeutic composition being incorporated into a pharmaceutically acceptable carrier, and said second therapeutic composition consisting essentially of a cholesterol replacement component, and at least one free fatty acid selected from the group consisting of essential free fatty acids and non-essential free fatty acids, the molar ratio of the cholesterol replacement component to the at least one free fatty acid being about 3:1; and
topically applying to the patient's skin a therapeutically effective amount of the other of said first therapeutic composition and a second therapeutic composition after a duration of at least several hours from the application of said one of said first therapeutic composition and said second therapeutic composition.

15. The method of claim 14, wherein said duration is a period of about twelve hours.

16. The method of claim 14, wherein the ceramide replacement component is a synthetic, non-animal derived analogue of ceramide.

17. The method of claim 16, wherein the synthetic, non-animal derived analogue of ceramide is bishydroxyethyl biscetyl malonamide.

18. The method of claim 14, wherein the cholesterol replacement component is a plant-derived analogue of cholesterol.

19. The method of claim 18, wherein the plant-derived analogue of cholesterol is at least one plant sterol.

20. The method of claim 18, wherein the plant-derived analogue of cholesterol is selected from the group consisting of phytosterols.

21. The method of claim 14, wherein the essential and non-essential free fatty acids are selected from the group consisting of arachidonic acid, linoleic acid, linolenic acid, palmitic acid, stearic acid, oleic acid, and docosanoic acid.

22. The method of claim 14, wherein the pharmaceutically acceptable carrier is suitable for topically applying the therapeutic composition to the skin and is selected from the group of creams, gels, lotions and ointments.

23. A composition for providing skin barrier lipids for skin moisturizing therapy, comprising:
bishydroxyethyl biscetyl malonamide as a ceramide replacement component;
a plant-derived analogue of cholesterol, selected from the group consisting of phytosterols as a cholesterol replacement component;
at least one essential free fatty acid; and
at least one non-essential free fatty acid, wherein the molar ratio of the bishydroxyethyl biscetyl malonamide, the plant-derived analogue of cholesterol, the at least one essential free fatty acid, and the at least one non-essential free fatty acid is approximately 3:1:1:1.

24. A composition for providing skin barrier lipids for skin moisturizing therapy, comprising:
bishydroxyethyl biscetyl malonamide as a ceramide replacement component;
a plant-derived analogue of cholesterol, selected from the group consisting of phytosterols as a cholesterol replacement component;
at least one essential free fatty acid; and
at least one non-essential free fatty acid, wherein the molar ratio of the bishydroxyethyl biscetyl malonamide, the plant-derived analogue of cholesterol, the at least one essential free fatty acid, and the at least one non-essential free fatty acid is approximately 1:3:1:1.

25. A composition for providing skin barrier lipids for skin moisturizing therapy, comprising:
bishydroxyethyl biscetyl malonamide as a ceramide replacement component;
a plant-derived analogue of cholesterol, selected from the group consisting of phytosterols as a cholesterol replacement component;
at least one essential free fatty acid; and
at least one non-essential free fatty acid, wherein the molar ratio of the bishydroxyethyl biscetyl malonamide, the plant-derived analogue of cholesterol, the at least one essential free fatty acid, and the at least one non-essential free fatty acid is approximately 1:1:1:1.
